(19) United States
(12) Patent Application Publication
(10) Pub. No.: US 2021/0077038 A1
(43) Pub. Date: Mar. 18, 2021

(54) X-RAY IMAGING DEVICE AND METHOD FOR IMAGING BY USING SAME

(71) Applicant: YOFO MEDICAL TECHNOLOGY CO., LTD., Hefei Anhui (CN)

(72) Inventor: Lei ZHU, Hefei Anhui (CN)

(21) Appl. No.: 16/954,873

(22) PCT Filed: Aug. 24, 2018
(86) PCT No.: PCT/CN2018/102048

§ 371 Date: Jun. 17, 2020

(30) Foreign Application Priority Data
Dec. 22, 2017 (CN) 201711402576.0

(51) Int. Cl. A61B 6/00

(52) U.S. Cl. CPC A61B 6/4007; A61B 6/4085; A61B 6/44; A61B 6/4208; A61B 6/4078

(57) ABSTRACT

The present application discloses an X-ray imaging device and a method of imaging using the X-ray imaging device. The X-ray imaging device comprises a rotating mechanism provided with a radiation source comprising a first radiation source and a second radiation source, the first radiation source and the second radiation source respectively emitting first light and second light, wherein the first light and the second light alternately irradiate a radiated body; and a detector for detecting the first light and the second light passing through the radiated body.

### CROSS REFERENCE TO RELATED APPLICATION

This application is a national application of PCT/CN2018/102048, filed on Aug. 24, 2018 and which claims priority to Chinese application 201711402576.0, filed on Dec. 22, 2017. The contents of PCT/CN2018/102048 and CN 201711402576.0 are all hereby incorporated by reference.

### TECHNICAL FIELD

The present application relates to the field of X-ray imaging, and more particularly, to an X-ray imaging device capable of expanding the scanning volume of a radiation source on a radiated body, and a method of imaging using the X-ray imaging device.

### BACKGROUND

Currently commonly used X-ray imaging devices, such as Computed Tomography devices (hereinafter referred to as CT devices) or chest DR, are typically provided with one radiation source and one detector, or two sets of scanning imaging systems, i.e. two radiation sources and two detectors. For example, a cone-beam computed tomography (CBCT) device is provided with one cone-beam source of radiation and a detector, and effective X-rays (i.e., X-rays that can be received by the detector) only reach limited areas due to limited size and shape of the detector. Further, since the adjustable distances among the radiation source, the radiated body, and the detector are limited, the scanning volume of the X-rays emitted by the radiation source is also limited. When a radiated body is relatively large in size, the X-rays that can be received by the detector cannot completely cover the part of the radiated body that needs to be scanned (hereinafter referred to as a region of interest), so that the entire scanned image of the region of interest cannot be obtained. For a CT device having two sets of scanning imaging systems, two sets of X-ray generating devices and two sets of detector systems are mounted at a certain angle on the same plane of gantry rotation to perform simultaneous scannings. Although spectral imaging can be achieved or temporal resolution can be improved, the longitudinal scanning volume is still limited. In addition, when two sets of scanning imaging systems are used to expand the longitudinal scanning volume, scattering between the X-rays generated by two radiation sources on the one hand would reduce the imaging resolution, and two detectors on the other hand would increase the manufacturing cost.

### SUMMARY

In order to overcome at least one of the above technical problems, the present application provides an X-ray imaging device comprising two radiation sources and one detector.

According to one aspect of the present application, there is provided an X-ray imaging device comprising: a rotating mechanism with a radiation source comprising a first radiation source and a second radiation source, wherein the first radiation source and the second radiation source respectively emit the first light and the second light, wherein the first light and the second light alternately irradiate a radiated body, wherein the rotating mechanism surrounds or partially surrounds the radiated body and is configured to rotate around the radiated body; and a detector for detecting the first light and the second light passing through the radiated body.

In one embodiment, the detector can be mounted on the rotating mechanism.

In one embodiment, the detector and the radiation source can be mounted on two sides of the radiated body.

In one embodiment, the first radiation source and the second radiation source can be pulsed radiation sources.

In one embodiment, the X-ray imaging device can further comprise a light switching mechanism comprising a baffle that alternately blocks the first light and the second light.

In one embodiment, the X-ray imaging device can further comprise a light switching mechanism comprising a first baffle and a second baffle, wherein the first baffle and the second baffle alternately block the first light and the second light, respectively.

In one embodiment, the first light and the second light can be cone beam X-rays or fan-shaped X-rays.

In one embodiment, the rotating mechanism surrounds or partially surrounds the radiated body and is configured to rotate about the radiated body.

In one embodiment, the first radiation source and the second radiation source can be spaced apart from each other in the direction of the rotational axis of the rotating mechanism or in the direction perpendicular to the rotational axis.

In one embodiment, the first radiation source and the second radiation source are arranged such that the first light and the second light scan the same portion on the radiated body in a single scanning, and wherein the first radiation source and the second radiation source are loaded with different tube voltages.

In one embodiment, waveforms of the first light and the second light are square wave, the first light and the second light have the same period, and the period is an even multiple of a detection period of the detector.

In one embodiment, the rotating mechanism can also be configured to move in the direction of the rotation axis thereof while rotating.

According to another aspect of the present application, there is provided a method of imaging using an X-ray imaging device as described above. The method comprises alternately irradiating a radiated body with the first light and the second light; and detecting the first light and the second light passing through the radiated body via the detector.

According to the X-ray imaging device as described above, by using the two radiation sources, not only can the scanning volume be increased, but also alternately irradiating with the first light and the second light can eliminate or partially eliminate resolution reduction caused by scattering of light generated by the two radiation sources. On the other hand, manufacturing cost is reduced by using only one detector. In addition, the X-ray imaging device according to the present application can also achieve spectral imaging by loading the first radiation source and the second radiation source with different tube voltages.

### BRIEF DESCRIPTION OF THE DRAWINGS

The above and other aspects, features and advantages of the present application will become more apparent by describing in detail exemplary embodiments thereof with reference to the accompanying drawings in which:

FIG. 1 is a schematic diagram of an X-ray imaging device according to an exemplary embodiment of the present application;

FIG. 2 is a schematic diagram of an X-ray imaging device according to an exemplary embodiment of the present application showing the arrangement of a radiation source and a detector and an optical path between the radiation source and the detector;

FIG. 3 is a schematic diagram of an X-ray imaging device according to another exemplary embodiment of the present application showing the arrangement of a radiation source and a detector and an optical path between the radiation source and the detector;

FIGS. 4 to 6 show a schematic diagrams of an embodiment in which the first radiation source 110 and the second radiation source 120 are pulsed radiation sources;

FIG. 7 is a schematic plan view showing the optical path among the first radiation source, the second radiation source, the detector and the radiated body as a whole when the rotating mechanism is rotating;

FIG. 8 is a schematic plan view independently showing an optical path among the first radiation source, the second radiation source, the detector, and the radiated body; and

FIG. 9 is a schematic diagram of an X-ray imaging device according to another exemplary embodiment of the present application showing the arrangement of a radiation source and a detector and an optical path between the radiation source and the detector.

### DETAILED DESCRIPTION

The present application will now be described more thoroughly hereinafter with reference to the accompanying drawings showing various embodiments. However, the present application can be implemented in multiple different forms and should not be construed as limited to the embodiments set forth herein. Rather, these embodiments are provided so that this disclosure will be thorough and complete, and will fully convey the scope of the present application to those skilled in the art. Throughout the specification and the drawings, the same reference numerals refer to the same elements.

It will be appreciated that when an element is said to be on another element, it can be directly on the other element, or intervening elements can be present therebetween. Conversely, when an element is said to be directly on another element, no intermediate element is present.

It will be understood that while the terms “first”, “second”, “third” and the like can be used herein to describe various elements, components, areas, layers, and/or portions, these elements, components, areas, layers, and/or portions should not be limited by these terms. These terms are used only to distinguish one element, component, area, layer, or portion from another element, component, area, layer, or portion. Thus, a first element, component, area, layer, or portion discussed below can be referred to as a second element, component, area, layer, or portion without departing from the teachings herein.

The terminology used herein is only for the purpose of describing particular embodiments and is not intended to be limiting. As used herein, the singular forms “a”, “an”, and “the” are intended to include the plural forms, comprising “at least one” unless indicated otherwise. As used herein, the phrase “and/or” includes any and all combinations of one or more of the relevant listed items. It will also be understood that the word “comprising” when used in this specification indicates the presence of stated features, areas, integers, steps, operations, elements, and/or components, but does not rule out the presence or addition of one or more other features, areas, integers, steps, operations, elements, components, and/or combination thereof.

In addition, the spatial relative terms, such as “below . . . ” or “above . . . ” and “on . . . ”, and the like, are used herein to describe the relationship of one element to another as shown in the drawings. It will be understood that in addition to the orientations described in the drawings, relative terms are also intended to encompass different orientations of the device. For example, if the device in one of the Figures is reversed, the element described as being “below” the other element will then be oriented to be “above” the other element. Exemplary terms “below” or “under” can thus encompass both the upper and lower orientations.

As used herein, “about” or “approximately” includes the stated values as well as averages within acceptable range of deviations for a particular value as determined by one of ordinary skill in the art in consideration of ongoing measurements and errors associated with a particular amount of measurements (i.e., limitations of the measurement system).

Unless defined otherwise, all terms (comprising technical and scientific terms) used herein have the same meaning as commonly understood by one of ordinary skill in the art to which this disclosure belongs. It will also be understood that terms, such as those defined in commonly used dictionaries, are to be interpreted as having a meaning consistent with their meaning in the context of the related art and in the present disclosure, and will not be interpreted in an idealized or overly formal sense unless expressly so defined herein.

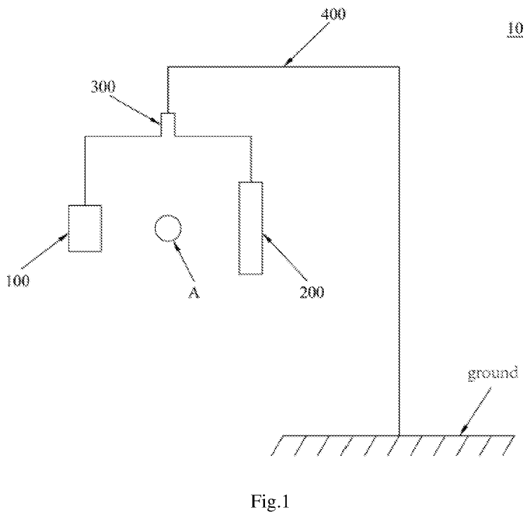

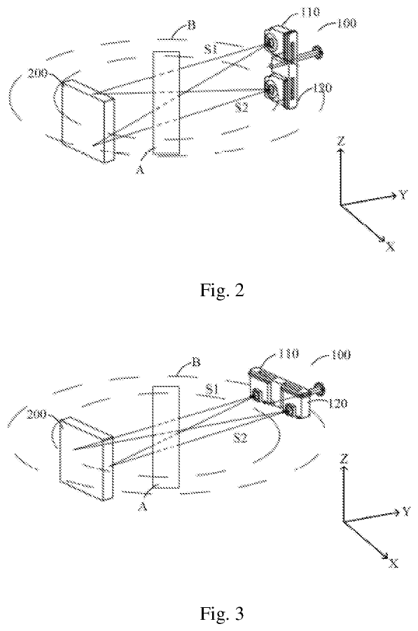

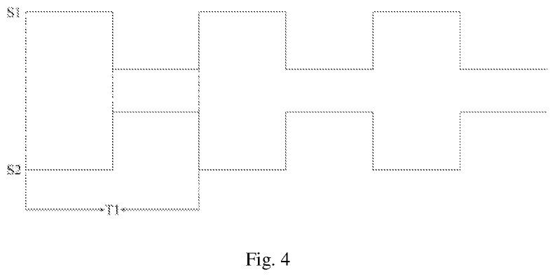

FIG. 1 is a schematic diagram of an X-ray imaging device 10 according to an exemplary embodiment of the present application. FIG. 2 is a schematic diagram of an X-ray imaging device 10 according to an exemplary embodiment of the present application showing the arrangement of a radiation source and a detector and an optical path between the radiation source and the detector. FIG. 3 is a schematic diagram of an X-ray imaging device 10 according to another exemplary embodiment of the present application showing the arrangement of a radiation source and a detector and an optical path between the radiation source and the detector.

Referring to FIGS. 1 to 3, the X-ray imaging device 10 according to the present application includes a rotating mechanism 300 provided with a radiation source 100 and a detector 200. The detector 200 can be a two-dimensional planar detector. For example, the detector 200 can be a flat panel detector, which can have a coverage width in the Z-axis direction of up to, for example, about 300 mm or more. In the embodiment shown in FIG. 1, the detector 200 can be provided on the rotating mechanism 300, but this is merely exemplary and the present application is not limited thereto. The detector 200 can also be provided on a mechanism other than the rotating mechanism 300, and can be configured to be capable of receiving the first light S1 and the second light S2 passing through a radiated body A. The radiation source 100 and the detector 200 can be provided on two sides of the radiated body A.

In the embodiment shown in FIG. 1, the rotating mechanism 300 can, for example, be connected to a frame 400, and the frame 400 can be fixedly mounted to or placed on the ground.

In one embodiment of the present application, a driving device (e.g., a motor) can be provided in the rotating mechanism 300, and the rotating mechanism 300 can be driven by the driving device to perform rotation or other movements, but this is merely exemplary. The driving device can be arranged in the rotating mechanism 300 or other suitable positions.

Referring to FIGS. 2 and 3, the radiation source 100 includes a first radiation source 110 and a second radiation source 120. The first radiation source 110 and the second radiation source 120 emit first light S1 and second light S2, respectively. The first radiation source 110 and the second radiation source 120 can be X-ray generators, and the first light S1 and the second light S2 can be X-rays. The first light S1 and the second light S2 can be cone beam X-rays, fan-shaped X-rays, or the like, but the present application is not limited thereto. By providing two radiation sources (i.e., the first radiation source 110 and the second radiation source 120), the scanning volume of the X-ray imaging device 10 can be expanded.

In the embodiment shown in FIG. 2, when the first radiation source 110 and the second radiation source 120 are spaced apart from each other in the Z-axis direction, the scanning volume of the first light S1 and the second light S2 on the radiated body A can be expanded in the longitudinal direction (i.e., the direction perpendicular to a plane B defined by the X-axis and the Y-axis, i.e., the Z-axis direction). In the embodiment shown in FIG. 3, when the first radiation source 110 and the second radiation source 120 are arranged side by side on, for example, the plane B formed by an X axis and a Y axis (i.e., on the plane B perpendicular to the Z axis), the scanning volume in a direction parallel to the plane B can be expanded. The arrangement of the radiation source 100 shown in FIGS. 2 and 3 is merely exemplary, and the present application is not limited thereto. Using different arrangements of the first and second radiation sources 110 and 120 in the radiation source 100 (i.e., different position relationships of the first and second radiation sources 110 and 120 with respect to the radiated body A), the expanding of the scanning volume in different directions can be realized, that is, the expanding of the scanning volume is not limited to that in the horizontal direction and the vertical direction.

In one embodiment of the present application, the X-ray imaging device 10 can further include a controller (not shown). The controller is able to control the switches of the radiation source 100, e.g., the controller can independently control the switches of the first radiation source 110 and the second radiation source 120. The controller can also be coupled to the driving device to control the rotational speed and direction of the driving device.

The first light S1 and the second light S2 alternately irradiate the radiated body A. The detector 200 detects the first light S1 and the second light S2 passing through the radiated body A. When the first light S1 and the second light S2 simultaneously irradiate the radiated body A, interference caused by scattering between the first light S1 and the second light S2 can result in reduced imaging accuracy. In the X-ray imaging device 10 according to the present application, the first light S1 and the second light S2 alternately irradiate the radiated body A. This can reduce the interference and/or scattering of the first light S1 and the second light S2 that occurs before or after the irradiation to the radiated body A. According to one embodiment of the present application, the first light S1 and the second light S2 can continuously and alternately irradiate the radiated body. According to another embodiment of the present application, the first light S1 and the second light S2 can alternately irradiate the radiated body A at a predetermined interval. In addition, the time at which the first light S1 and the second light S2 irradiate the radiated body A can be selected as required.

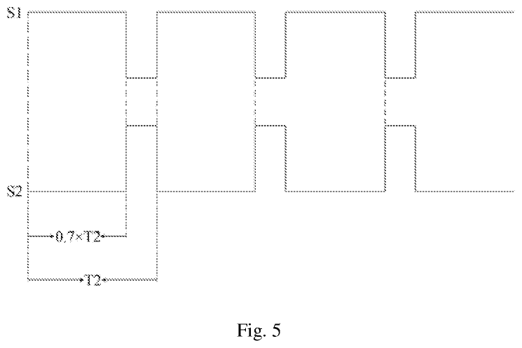

FIGS. 4 to 6 are exemplary waveform diagrams of the first light S1 and the second light S2 where the first radiation source 110 and the second radiation source 120 are pulsed radiation sources.

According to one embodiment of the present application, the first radiation source 110 and the second radiation source 120 can be pulsed radiation sources, i.e., the first radiation source 110 and the second radiation source 120 can emit X-ray pulses. For example, the first radiation source 110 and the second radiation source 120 can be pulsed lasers, and the waveforms of the first light S1 and the second light S2 can be rectangular waves. The first radiation source 110 and the second radiation source 120 can be configured such that the first light S1 and the second light S2 alternately irradiate the radiated body A. The intensities of the first light S1 and the second light S2 can be identical or different.

Referring to FIG. 4, according to an alternative embodiment of the present application, the first light S1 and the second light S2 emitted by the first radiation source 110 and the second radiation source 120, respectively, can have the same period T1, and the duty ratios of the first light S1 and the second light S2 can each be 50%, that is, the first light S1 and the second light S2 can be square waves. The first radiation source 110 and the second radiation source 120 can be configured such that the second light S2 is at the falling edge when the first light S1 is at the rising edge, and the first light S1 is at the falling edge when the second light S2 is at the rising edge, that is, the first light S1 and the second light S2 do not simultaneously irradiate the radiated body A. In this case, the period T1 of the first light S1 or the second light S2 can be an even multiple of the detection period of the detector 200. The detection period of the detector 200 refers to the time required for the detector 200 to complete a detection. In this way, it is convenient to distinguish the data acquired by the detector 200, for example, to distinguish the data generated by the detector 200 after the first light S1 irradiates the radiated body A from the data generated by the detector 200 after the second light S2 irradiates the radiated body A.

Referring to FIG. 5, the embodiment of FIG. 5 is generally identical to the embodiment of FIG. 4 except for the duty ratios of the first light S1 and the second light S2. The first light S1 and the second light S2 can have the same period T2, the first light S1 and the second light S2 have different duty ratios and the sum of the duty ratios of the first light S1 and the second light S2 is 1. For example, the duty ratio of the first light S1 can be 70%, and the duty ratio of the second light can be 30%, but the present application is not limited thereto. In this embodiment, the duty ratios of the first light S1 and the second light S2 can be greater than 0 and less than 100%, but not equal to 50%. The second light S2 is at a falling edge when the first light S1 is at a rising edge, and the second light S2 is at a rising edge when the first light S1 is at a falling edge. That is, the first light S1 and the second light S2 irradiate the radiated body A at different time. In one example, it can be determined from the duty ratios of the first light S1 and the second light S2 that the light detected by the detector 200 during a first predetermined time is the first light S1 passing through the radiated body A and the light detected during a second predetermined time is the second light S2 passing through the radiated body A. The first predetermined time and the second predetermined time can be determined according to the duty ratios of the first light S1 and the second light S2, that is, according to the time for which the first light S1 and the second light S2 irradiate the radiated body A in one period T2. For example, if the duty ratio of the first light S1 is 70% and the duty ratio of the second light is 30%, it can be determined that the light detected by the detector 200 within the time period of 0.7×T2 after the start of the detection is the first light S1 passing through the radiated body A, and it can be determined that the light detected by the detector 200 within the next time period of 0.3×T2 is the second light S2 passing through the radiated body A. The above embodiments facilitate distinguishing between data acquired by the detector 200 and are merely exemplary, and the present application is not limited thereto.

Referring to FIG. 6, the first light S1 and the second light S2 can have the same period T3 and the sum of the duty ratios of the first light S1 and the second light S2 is less than 1. For example, the duty ratio of the first light S1 is 70%, and the duty ratio of the second light S2 is 20%. In the embodiment of FIG. 6, the first light S1 is at the rising edge at first, that is, the first light S1 irradiates first, and the second light S2 is also at the falling edge when the first light S1 is at the falling edge. At this time, neither the first light S1 nor the second light S2 irradiates the radiated body A. In the case where the duty ratio of the first light S1 is 70% and the duty ratio of the second light S2 is 20%, the time for which neither the first light S1 nor the second light S2 irradiates the radiated body A in one period T3 can last for 0.1×T3. In one period T3, the second light S2 can irradiate for a time of 0.2×T3. The above process can be cycled as required by the scan. The above embodiments facilitate distinguishing between data acquired by the detector 200 and are merely exemplary, and the present application is not limited thereto.

The above-described embodiments are merely exemplary. The first light S1 and the second light S2 can be aperiodic pulses. In this case, the first radiation source 110 and the second radiation source 120 can be configured such that the first light S1 and the second light S2 alternately irradiate the radiated body A, that is, the first light S1 and the second light S2 do not simultaneously irradiate the radiated body A.

According to another embodiment of the present application, the X-ray imaging device 10 can include a light switching mechanism, which can comprise a baffle that alternately blocks the first light S1 and the second light S2. This allows the first light S1 and the second light S2 to alternately irradiate the radiated body A. The light switching mechanism can be controlled, for example, by a controller included in the X-ray imaging device 10. For example, when the X-ray imaging device 10 comprises a computer system, the light switching mechanism can be controlled by a controller comprised in the computer system, but the present application is not limited thereto.

According to yet another embodiment of the present application, the X-ray imaging device 10 can include a light switching mechanism comprising a first baffle and a second baffle, wherein the first baffle and the second baffle alternately block the first light S1 and the second light S2, respectively. This allows the first light S1 and the second light S2 to alternately irradiate the radiated body A. The light switching mechanism can be controlled, for example, by a controller included in the X-ray imaging device 10. For example, when the X-ray imaging device 10 comprises a computer system, the light switching mechanism can be controlled by a controller comprised in the computer system, but the present application is not limited thereto.

The above-described embodiments are merely exemplary, and alternate irradiation on the radiated body A by the first light S1 and the second light S2 can be achieved in a manner other than the embodiments described above.

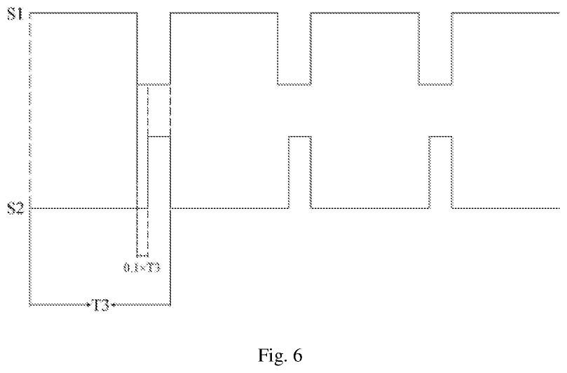

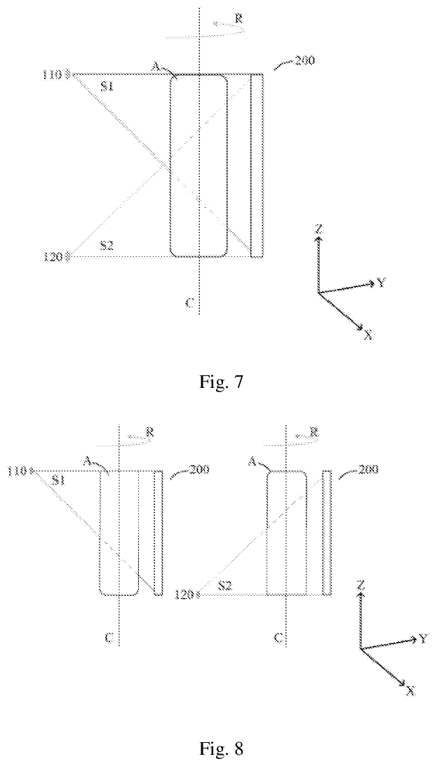

FIG. 7 is a schematic plan view showing the optical path among the first radiation source, the second radiation source, the detector and the radiated body as a whole when the rotating mechanism is rotating. FIG. 8 is a schematic plan view independently showing an optical path among the first radiation source, the second radiation source, the detector, and the radiated body.

According to one embodiment of the present application, the rotating mechanism 300 can surround or partially surround the radiated body A, and can be configured to rotate about the radiated body A. When the rotating mechanism 300 surrounds the radiated body A, it can be in a closed loop, but the shape of the rotating mechanism 300 is not limited thereto. When the rotating mechanism 300 partially surrounds the radiated body A, it can be in an annular shape having an opening, a semi-circular shape, a C-shape, or the like, but the shape of the rotating mechanism 300 is not limited thereto. As shown in FIGS. 7 and 8, the rotation axis C of the rotating mechanism 300 can be overlapped with the radiated body A, but the present application is not limited thereto. The rotation axis C can be arranged at other positions. The rotation direction R of the rotating mechanism 300 is not limited to the directions shown in FIGS. 7 and 8, and can be opposite to the directions shown in FIGS. 7 and 8 or can be oblique with respect to the radiated body A. The speed of the rotating mechanism 300 about the radiated body A can be set according to imaging requirements.

As shown in FIGS. 7 and 8, when the first radiation source 110 and the second radiation source 120 are spaced apart in the direction of the rotation axis C, the first light S1 can irradiate a portion of the radiated body A (e.g., about an upper ¾ portion of the radiated body A), and the second light S2 can irradiate a portion of the radiated body A (e.g., about a lower ¾ portion of the radiated body). Accordingly, the first light S1 and the second light S2 can scan the entire radiated body in a single scan, thereby expanding the scanning volume of the X-ray imaging device 10 in the longitudinal direction (i.e., the direction of the rotation axis C). A single scan can refer to an action of the X-ray imaging device according to the present application to complete scanning of an area to be imaged on the radiated body A. The action can be set as required, for example, the scanning time and the scanning number of the first light S1 and the second light S2, the time interval between the first light S1 and the second light S2, and the scanning direction and the scanning speed of the first light S1 and the second light S2 (for example, by setting the rotation direction and the rotation speed of the rotating mechanism 300) can be set as required. The action can be, for example, scanning the area to be imaged twice with the first light S1 for 45 seconds each time, and scanning the area to be imaged with the second light S2 for 60 seconds before the second scanning with the first light S1, and the scanning interval of 5 seconds between the first light S1 and the second light S2. However, this is merely exemplary, and the present application is not limited thereto.

In the case where the rotating mechanism 300 is rotatable about the radiated body A, the first radiation source 110 and the second radiation source 120 can also be spaced apart from each other in a direction perpendicular to the rotation axis C, but the present application is not limited thereto.

By rotating the rotating mechanism 300 provided with the first radiation source 110 and the second radiation source 120 spaced apart in the direction of the rotation axis C about the radiated body A, for example, the scanning volume of the X-ray imaging device 10 in the longitudinal direction (in the direction of the rotation axis C) can be expanded while volume scanning is achieved. In addition, since the first light S1 and the second light S2 alternately irradiate the radiated body A, scattering or interference generated by the simultaneous irradiation of the first light S1 and the second light S2 can also be avoided.

The detector 200 can be provided on the rotating mechanism 300 together with the radiation source 100, and the detector 200 and the radiation source 100 are provided on two sides of the radiated body A, respectively. The detector 200 and the radiation source 100 are thus rotatable about the radiated body A with the rotating mechanism 300.

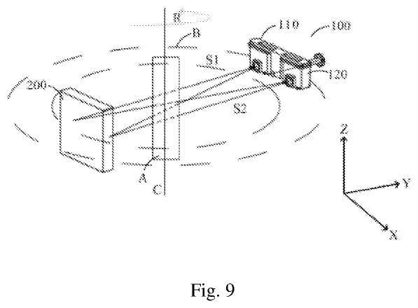

FIG. 9 is a schematic diagram of an X-ray imaging device according to another exemplary embodiment of the present application showing the arrangement of a radiation source and a detector and an optical path between the radiation source and the detector.

According to another embodiment of the present application, the rotating mechanism 300 can surround or partially surround the radiated body A, and are configured to rotate about the radiated body A. For example, the rotating mechanism 300 can be driven by a driving device provided in the X-ray imaging device 10. It is well-known in the prior art how the driving device drives the rotating mechanism 300 to rotation or perform other movements, and therefore they are not described in detail. The first radiation source 110 and the second radiation source 120 can be disposed on a rotating mechanism. The first radiation source 110 and the second radiation source 120 can be configured such that the first light S1 and the second light S2 scan the same portion on the radiated body A in a single scan, wherein a single scan can refer to an action of the X-ray imaging device according to the present application to complete scanning of an area to be imaged on the radiated body A, as described above with reference to FIGS. 7 and 8. The same portion can be the image acquisition area of the X-ray imaging device 10 for the radiated body A. In the embodiment shown in FIG. 9, the first radiation source 110 and the second radiation source 120 can, for example, be arranged spaced apart from each other in a direction parallel to the plane B. In this case, the first light S1 and the second light S2 can irradiate at the same height of the radiated body A with respect to the plane B. Thus, in a single scan in which the first radiation source 110 and the second radiation source 120 rotate together with the rotating mechanism 300, the first light S1 and the second light S2 scan the same portion on the radiated body A. Scanning the same portion on the radiated body A with the first light S1 and the second light S2 can indicate that the first light S1 and the second light S2 overlap completely or partially in a single scanning with respect to the scanning portion on the radiated body A (i.e., the portion where the volume scan is achieved). When the rotating mechanism 300 surrounds the radiated body A, it can be a closed ring, but the shape of the rotating mechanism 300 is not limited thereto. When the rotating mechanism 300 partially surrounds the radiated body A, it can be an annular shape having an opening, a semi-circular shape, a C-shape, or the like, but the shape of the rotating mechanism 300 is not limited thereto.

In the embodiment shown in FIG. 9, the first radiation source 110 and the second radiation source 120 can be loaded with different tube voltages. For example, the first radiation source 110 can be loaded with a high tube voltage, and the second radiation source 120 can be loaded with a low tube voltage. The data corresponding to the first radiation source 110 detected by the detector 200 can be a high-energy signal, and the data corresponding to the second radiation source 120 detected by the detector 200 can be a low-energy signal. Based on the obtained high-energy signal and the low-energy signal, a spectral CT image of the irradiated portion of the radiated body A can be obtained by an image reconstruction algorithm. Therefore, spectral X-ray imaging can be realized by the X-ray imaging device 10 according to the present embodiment. In a spectral CT having a single radiation source, it is necessary to scan an image acquisition area by a single radiation source to which different tube voltages are applied. The time resolution of the spectral CT is low, so that the use of spectral CT is limited. The X-ray imaging device 10 according to the present application uses two radiation sources, so that the scanning of the same part (image acquisition area) on the radiated body A can be achieved by a single scan, thereby realizing the image acquisition in a short time.

According to an embodiment of the present application, in the embodiment shown in FIGS. 7 to 9, the rotating mechanism 300 can also be configured to move in the direction of the rotation axis of the rotating mechanism 300 while rotating about the rotation axis C. By moving the rotating mechanism 300 in the direction of the rotation axis C, for example, upward (Z-axis direction) or downward (opposite direction of the Z-axis), the scanning volume of the X-ray imaging mechanism can be further expanded.

The position relationships of the first radiation source 110 and the second radiation source 120 are not limited to the embodiments described above. The first radiation source 110 and the second radiation source 120 can be integrally formed, detachably connected, or separately formed, but the present application is not limited thereto.

In FIGS. 2, 3, and 7 to 9, the first light S1 and the second light S2 are schematically shown by two lines, respectively, for ease of description, but the first light S1 and the second light S2 are not limited to the form shown in the Figures.

According to another aspect of the present application, the present application also discloses a method of imaging by the X-ray imaging device 10 as described above. The method includes alternately irradiating the radiated body with the first light and the second light; and detecting the first light and the second light passing through the radiated body via the detector.

By means of the X-ray imaging device of the present application and the method of imaging using the same, the scanning volume of the X-rays, for example, a lateral (e.g., a direction parallel to the plane B) scanning volume and a longitudinal (Z-axis direction) scanning volume, can be expanded. In addition, when the X-ray imaging device 10 is provided with the first light source 110 and the second light source 120 spaced apart from each other in the longitudinal direction (for example, the Z-axis direction), and when the X-ray imaging device 10 is used and the rotating mechanism 300 rotates around the radiated body A, the volume scanning of the radiated body A can be achieved while the scanning volume is expanded. In the case where the rotating mechanism 300 is rotatable about the radiated body A, the X-ray imaging device can realize spectral X-ray imaging by loading the first radiation source 110 and the second radiation source 120 with different tube voltages.

While certain exemplary embodiments and examples have been described herein, other embodiments and modifications will be apparent from the above descriptions. Various changes and modifications can be made to the embodiments of the present application by those skilled in the art without departing from the teachings of the present application. Accordingly, the inventive concept is not limited to these embodiments, but is defined by the appended claims and the broader scope of various obvious modifications and equivalent arrangements.

What is claimed is:

1. An X-ray imaging device comprising:
a rotating mechanism provided with a radiation source comprising a first radiation source and a second radiation source, the first radiation source and the second radiation source respectively emitting first light and second light, wherein the first light and the second light alternately irradiate a radiated body; and
a detector for detecting the first light and the second light passing through the radiated body.

2. The X-ray imaging device according to claim 1, wherein the detector is mounted on the rotating mechanism.

3. The X-ray imaging device according to claim 2, wherein the detector and the radiation source are mounted on two sides of the radiated body.

4. The X-ray imaging device according to claim 1, wherein the first radiation source and the second radiation source are pulsed radiation sources.

5. The X-ray imaging device according to claim 1, further comprising a light switching mechanism comprising a baffle that alternately blocks the first light and the second light.

6. The X-ray imaging device according to claim 1, further comprising a light switching mechanism comprising a first baffle and a second baffle, wherein the first baffle and the second baffle alternately block the first light and the second light, respectively.

7. The X-ray imaging device according to claim 1, wherein the first light and the second light are cone beam X-rays or fan-shaped X-rays.

8. The X-ray imaging device according to claim 1, wherein the rotating mechanism surrounds or partially surrounds the radiated body and is configured to rotate about the radiated body.

9. The X-ray imaging device according to claim 8, wherein the first radiation source and the second radiation source are spaced apart from each other in the direction of the rotational axis of the rotating mechanism or in the direction perpendicular to the rotational axis.

10. The X-ray imaging device according to claim 8, wherein the first radiation source and the second radiation source are arranged such that the first light and the second light scan the same portion on the radiated body in a single scanning, and wherein the first radiation source and the second radiation source are loaded with different tube voltages.

11. The X-ray imaging device according to claim 4, wherein waveforms of the first light and the second light are square waves, the first light and the second light have the same period, and the period is an even multiple of a detection period of the detector.

12. The X-ray imaging device according to claim 1, wherein the rotating mechanism is further configured to move in the direction of the rotation axis thereof while rotating.

13. A method of imaging using an X-ray imaging device,
wherein the X-ray imaging device comprises:
a rotating mechanism provided with a radiation source comprising a first radiation source and a second radiation source, the first radiation source and the second radiation source respectively emitting first light and second light, wherein the first light and the second light alternately irradiate a radiated body; and
a detector for detecting the first light and the second light passing through the radiated body; and
wherein the method of imaging comprises:
alternately irradiating a radiated body with the first light and the second light; and
detecting the first light and the second light passing through the radiated body via the detector.

14. The method of imaging using an X-ray imaging device according to claim 13,
wherein the X-ray imaging device further comprises a light switching mechanism comprising a baffle; and
wherein the method of imaging further comprises the baffle alternately blocking the first light and the second light.

15. The method of imaging using an X-ray imaging device according to claim 13,
wherein the X-ray imaging device further comprises a light switching mechanism comprising a first baffle and a second baffle; and
wherein the method of imaging comprises the first baffle and the second baffle alternately blocking the first light and the second light, respectively.

16. The method of imaging using an X-ray imaging device according to claim 13,
wherein the rotating mechanism of the X-ray imaging device surrounds or partially surrounds the radiated body and is configured to rotate about the radiated body; and
wherein the method of imaging further comprises:
rotating the rotating mechanism around the radiated body.

17. The method of imaging using an X-ray imaging device according to claim 16, wherein
the first light and the second light scan the same portion on the radiated body in a single scanning, and
the first radiation source and the second radiation source are loaded with different tube voltages.

18. The method of imaging using an X-ray imaging device according to claim 13, further comprising moving the rotating mechanism in the direction of the rotation axis thereof while rotating.
